(19) United States
(12) Patent Application Publication
(10) Pub. No.: US 2004/0126874 A1
(43) Pub. Date: Jul. 1, 2004

(54) Cone plate type blood or the like compatibility evaluation device

(76) Inventors: Katsuko Sakai, Tokyo (JP); Tetsuya Tateishi, Ibaraki (JP); Takashi Ushida, Ibaraki (JP)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/466,711

(22) PCT Filed: Jan. 18, 2002
(86) PCT No.: PCT/JP02/00321

(30) Foreign Application Priority Data
Jan. 18, 2001 (JP) 200110706

(51) Int. Cl. C12M 1/34

(52) U.S. Cl. 435/287.1

(57) ABSTRACT

A cone-plate type apparatus for evaluating compatibility with blood or the like includes: a bottom plate (1) that secures test material (2), a cone (16) made of transparent material that rotates while in contact with blood or the like supplied upon the test material, a cone holder (13) that secures the cone therein and that has a rotary driver (14) on its periphery, a driving apparatus (8) that rotates the cone holder via the rotary driver and an incident light fluorescent microscope (2) for observing blood or the like on the test material from above the cone. The test material is secured to the bottom plate by means of ring members so it is possible to observe and quantitatively evaluate the state of adherence of blood cell components to not only plate-shaped material but also materials having the shape of a membrane, sponge, filter or any other shape, along with opaque materials, in the presence of shear stresses and in real time.

### TECHNICAL FIELD

The present invention relates to a device used to analyze and observe interactions between blood cell components and materials that make up artificial hearts and blood vessels in the presence of shear stress, and more specifically relates to a cone-plate type apparatus for evaluating compatibility with blood or the like that uses a rotating cone to generate shear stress and thus permits real-time analysis of interactions between various opaque materials in the form of plates, membranes, sponges, filters or other shapes and blood cell components of blood or blood cell suspensions (hereinafter referred to as “blood or the like”) in the presence of shear stress and evaluate compatibility with blood or the like.

### BACKGROUND ART

The constituent materials for implanted artificial hearts and artificial blood vessels and the like are required to have compatibility with respect to blood or the like and have the function of preventing coagulation (thrombus) or breakdown (hemolysis) of the blood or the like.

As a technique for evaluating antithrombogenicity or the ability to prevent thrombus formation, a method whereby the material is placed in contact with blood for a fixed amount of time and the number of blood platelets serving as triggers for thrombus formation that adhere to the surface of the material is quantitatively analyzed has been long accepted. However, within the body, blood platelets adhere to material and thus thrombi are formed in the presence of a flow, so there have been problems with the reliability of the data obtained by this technique. Moreover, problems also remain with the reproducibility of data.

As a technique for evaluating antithrombogenicity in the presence of shear stress present within the body, many researchers have adopted a technique called the column bead method. In this technique, glass beads or the like coated with the material being tested are packed into a column through which blood is circulated and then the number of blood platelets adhering to the material is quantitatively analyzed. This technique has a problem in that it is limited to testing materials that can be formed into bead shapes or materials that can be coated onto beads, a problem in that turbulent flows not present within the normal body are present around the beads among other problems.

As a technique for quantitatively analyzing the number of blood platelets adhering in the presence of the laminar flow present within a normal blood vessel, parallel plate type shear stress loading apparatus has been developed. With this apparatus, a blood plasma suspension is allowed to flow between two plate-shaped pieces of material so a laminar flow can be easily applied to the material surface, but a pump or the like must be used to eject the blood platelet suspension, so there have been problems in the data with artifacts arising from activation of the blood platelets arising at the time that the pump pushes fluid out and from the pump materials.

As a means of achieving laminar flow over the surface of the material in a simple system without using a pump or the like, a cone-plate type shear stress loading apparatus has previously been reported. This apparatus is characterized in that an inverted circular cone-shaped cone is rotated on the surface of the material, thus conveniently loading the surface of the material with a uniform shear stress.

With such a shear loading apparatus, a cone-plate type shear stress loading apparatus wherein blood or the like is placed upon a plate-shaped test piece and the circular cone-shaped cone is brought into contact with the blood or the like from above and rotated in this state is used, and by using a microscope to observe the state of the blood or the like during this rotation, the interaction between the blood cell components and various materials can be observed. The observation apparatus used here consists of a commercial rheometer that has been modified so that a plate-shaped piece of material can be secured using negative pressure and a commercial inverted microscope.

With such an apparatus for the observation of blood cell components on material surfaces consisting of a combination of a microscope and a conventional cone-plate type shear stress loading apparatus, a commercial cone-plate type shear stress loading apparatus used to measure the viscosity of blood or the like is used, so at the time of rotating the cone with a motor, a direct-drive mechanism wherein the cone is driven directly by a motor disposed above had been used.

For this reason, it is not possible to observe the appearance of the blood or the like upon the test material from above the cone, so it was necessary to observe the blood or the like on the surface of the material from beneath the material. For this reason, the only materials that can be observed in the conventional apparatus are extremely transparent materials that allow sufficient light to pass through, so there was a drawback in that the materials subject to evaluation were limited.

However, a large portion of the medical materials actually developed are opaque so there was a problem that such a conventional apparatus is not suitable as a general-purpose typical apparatus for observing the interactions between materials and blood plasma or blood cell components.

Moreover, not only a large portion of the test materials is opaque as described above, but also not all materials are plate-shaped, but rather they may take the form of membranes, sponges, filters or various other forms. However, with such a conventional cone-plate type shear stress loading apparatus the material is secured to the apparatus by negative pressure, so its application is limited to plate-shaped materials and here is a problem in that it is not applicable to materials having other shapes.

Accordingly, the present invention has as its object to provide a general-purpose compatibility evaluation apparatus that can be used to observe the interaction between blood cell components and not only plate-shaped materials but also materials having the shape of a membrane, sponge, filter or any other shape, along with opaque materials, in the presence of shear stresses and in real time.

### DISCLOSURE OF THE INVENTION

The present invention provides a cone-plate type apparatus for evaluating compatibility with blood or the like characterized in comprising: a bottom plate that secures test material, a cone made of transparent material that rotates while in contact with blood or the like supplied upon the test material, a cone holder that secures the cone therein and that has a rotary driver on its periphery, a driving apparatus that rotates the cone holder via the rotary driver and an incident light microscope for observing blood or the like on the test material from above the cone.

The rotary driver of the cone holder is a ring gear and the ring gear is rotary driven by a gear of the driving apparatus via an intermediate gear.

Each of the gears is supported at a stipulated position via bearings.

The bearings are air bearings that use air supplied from an air compressor.

The test material is in the form of a plate, membrane, sponge, filter or any other shape and is secured to the bottom plate by means of a ring member provided with an O-ring shaped elastic ring.

The bottom plate has a plurality of holes, and in each of the holes is installed an access port that is able to supply blood or the like to the surface of the test material in the presence of shear stress due to the rotation of the cone.

The cone makes a small angle with respect to the test material so that the interaction between the test material and blood cell components can be examined with a small quantity of blood or the like.

It comprises a height adjustment apparatus that adjusts the height of the cone with respect to the bottom plate.

The microscope has an objective lens disposed above the cone.

The cone is secured by means of a ring to the cone holder.

A gasket is provided between the cone and cone holder, thus preventing leakage of blood or blood platelet suspension.

A linkage screw is provided between the cone holder and the driving apparatus that drives the cone holder.

A moveable arm that secures the bottom plate and also rotatably supports the cone holder such that the cone is able to oscillate freely between directly below the microscope and other portions.

A translation apparatus that focuses the microscope on the surface of the test material is provided in the driving apparatus area.

The driving apparatus is provided with a motor that can load a superposition strain.

It comprises an X-Y-Z axial movement mechanism that changes the observation position on the surface of the test material.

The microscope is an incident light fluorescent microscope provided with a super-long focal length objective lens for observing the surface of the test material from above.

The microscope is provided with an illumination apparatus that projects light from the front side of the test material and an illumination apparatus that projects light from the rear side thereof.

The apparatus is entirely enclosed within a box provided with a temperature control apparatus for controlling the temperature of the interior at a fixed temperature.

The driving apparatus and microscope are installed upon a unitary vibration proof plate.

It is provided with an adjustment mechanism that adjusts the relative position of the observation apparatus consisting of the microscope and a shear stress measuring apparatus.

The driving apparatus is connected to a computer that controls the rotation of the cone.

The incident light microscope is provided with a super high-sensitivity camera connected to an image analyzer, timer, video recorder and monitor.

As described above, the apparatus for evaluating compatibility according to the present invention has the objective lens of the microscope placed above the transparent cone, so observation from above is possible, and thus even opaque test materials can be evaluated accurately. Moreover, the test material is secured to the bottom plate by means of a holder ring so it is possible to observe and evaluate the state of adherence of blood cell components to not only plate-shaped material but also materials having the shape of a membrane, sponge, filter or any other shape, along with opaque materials, in the presence of shear stresses and in real time.

### BRIEF DESCRIPTION OF THE DRAWINGS

FIG. 1 is an overall structural diagram of the cone-plate type apparatus for evaluating compatibility with blood or the like according to the present invention which is equipped with a shear stress loading apparatus and incident light fluorescent microscope.

FIG. 2(a) is a top view of a holder ring used to hold test material.

FIG. 2(b) is a top view of one example of test material.

FIG. 2(c) is a bottom view of the bottom plate.

FIG. 2(d) is a partial-cutaway top view of the test material secured to the bottom plate.

FIG. 3 is a cutaway side view with a partial cutaway showing the test material mounted on the bottom plate and the cone installed.

### BEST MODE FOR CARRYING OUT THE INVENTION

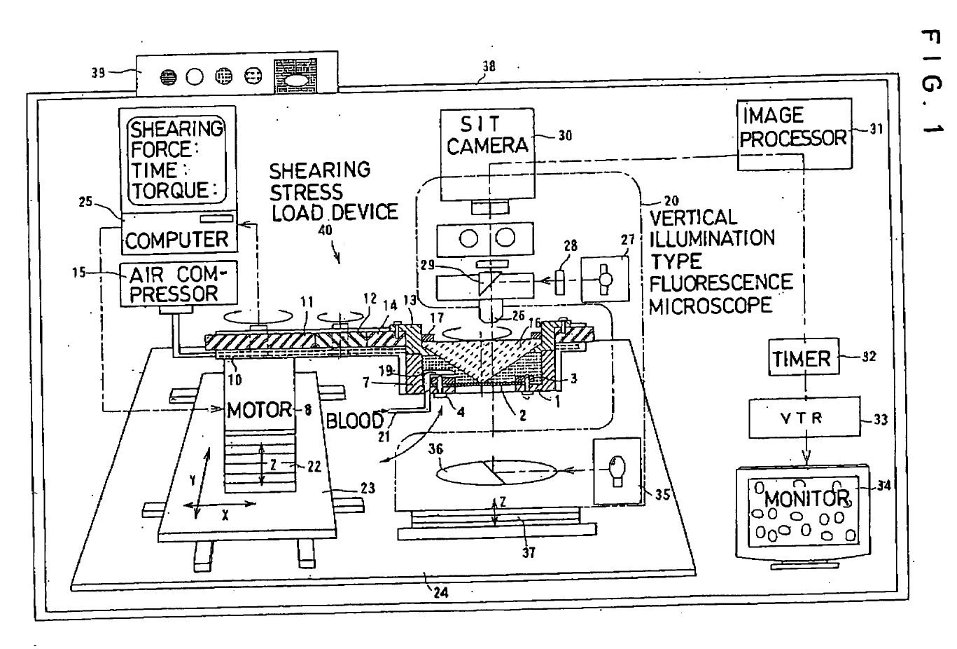

FIG. 1 shows a schematic view of the overall configuration of the cone-plate type apparatus for evaluating compatibility with blood or the like according to the present invention. Test material 2 is placed upon a bottom plate 1 that has an opening in its center for exposing the test material 2 as described later. The periphery of the test material is held down with a holder ring 3 provided with an O-ring or other elastic ring. Bolts 4 inserted from the bottom surface of the bottom plate 1 are used to secure the test material 2 to the bottom plate 1 together with the holder ring 3. The peripheral surface upon the bottom plate has a cylindrical support 7, and in the illustrated working example, this support 7 is provided on the end of a support plate 10 that is rotatably mounted to the frame of a motor 8. Screws or other means can be adopted as the means of securing the support 7 to this support plate 10.

The gear 11 of the motor 8 is engaged to an intermediate gear 12, and this intermediate gear 12 is engaged to a ring gear 14 secured by bolt to the periphery of a cylindrical cone holder 13. The intermediate gear 12 and ring gear 14 secured to the cone holder 13 in particular are supported upon the support plate 10 via bearings. In the illustrated working example, the gears are able to rotate with virtually no resistance due to air bearings that use air supplied from an air compressor 15.

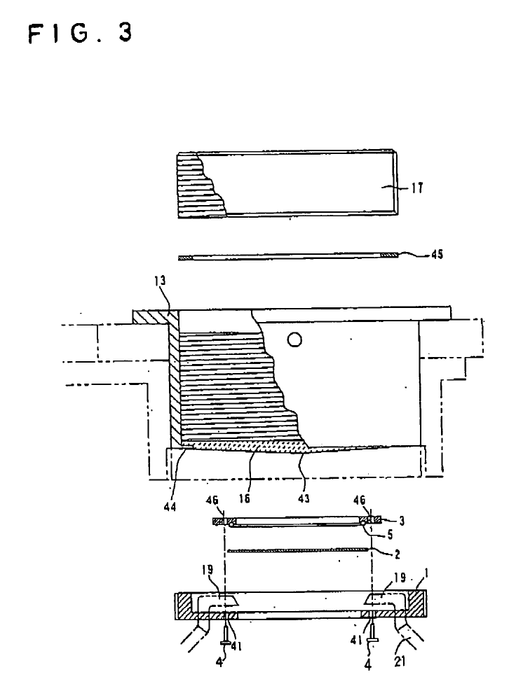

A transparent cone 16 is mounted in the cone holder 13 and the upper part of its periphery is secured by being held with a ring 17 via a gasket or other sealing apparatus 45. FIG. 1 is a schematic view of the cone 16, but in fact, the cone portion of this cone 16 makes a smaller angle with the test material in the plate portion, being nearly planar (FIG. 3). The tip of the cone is in proximity to the test material 1 and comes into contact with the blood or the like supplied upon the test material as it is rotated by the motor 8, thereby applying shear force to the blood or the like.

In the illustrated working example, the support plate 10 is secured to the frame of the motor 8 as described above, and is able to rotate by roughly 90 degrees about the driveshaft of the motor 8 so that the gap between the tip of the cone and the support plate can be set to be constant. It is rotated and moved forward at the time of performing the work of mounting test material and adjusting the cone, and thereafter, it can be returned to the stipulated position of the upright incident light fluorescent microscope 20 as shown in the figure. In addition in the illustrated working example, an access port 19 is provided to the bottom plate 1 upon which the test material 2 is placed so that blood or the like can be supplied upon the test material 2 mounted as described above, and a supply tube 21 is extended to and inserted into the access port 19.

The motor 8 is supported by a height adjustment apparatus 22 that uses a piezoelectric device or the like to focus the microscope, while the motor and height adjustment apparatus are secured upon an X-Y stage 23. The X-Y stage 23 is secured upon a vibration proof plate 24 such that it is free to move in the direction of the X-axis and the direction of the Y-axis, and thus the location viewed by the microscope can be freely adjusted. The motor 8 which is able to load superposition strain is connected to a computer 25, and thus its rotation can be controlled to a stipulated state and also the computer 25 is able to detect the torque of the motor 8 and also detect the load received due to agitation of the blood or the like when the cone 16 rotates. Thereby, the computer 25 calculates the shear force on the blood or the like and displays it on a display or outputs it to a printer or the like. These pieces of apparatus thus constitute a cone-plate type shear stress loading apparatus 40.

On the other hand, a super-long focal length objective lens 26 within the upright incident light fluorescent microscope 20 is positioned above the transparent cone 16, and the light from a mercury lamp 27 is able to illuminate the test material 2 via a filter 28, half mirror 27, objective lens 26 and the cone 16, respectively. In addition, the state of the blood or the like upon the test material 2 thus illuminated can be photographed with a high-precision SIT camera 30 via the objective lens 26 and half mirror 29. The images are subjected to image analysis and image processing by an image processor 31 and output to an image recording apparatus equipped with a timer 32, VCR 33 and the like and the state can also be displayed on a monitor 34.

As described above, the objective lens 26 of the upright incident light fluorescent microscope 20 is positioned above the cone 16 so not only transparent material but also opaque material can be evaluated accurately.

If necessary, this upright incident light fluorescent microscope 20 can also be used as a transmission microscope by passing the light of a halogen lamp 35 through a condenser 36 so that it illuminates the test material 2 from the rear side. In addition, a height adjustment apparatus 37 is provided in the base of the microscope so that it is possible to adjust the height of the shear stress loading apparatus 40 and microscope 20.

These pieces of apparatus thus constitute a cone-plate type apparatus for evaluating compatibility with blood or the like that can be used to observe the actual state of blood or the like corresponding to measurement with the cone-plate type shear stress loading apparatus, and these pieces of apparatus are secured upon a unitary vibration proof plate 24 and also enclosed within a box 38. This box 38 is controlled to a stipulated temperature by means of a temperature control apparatus 39.

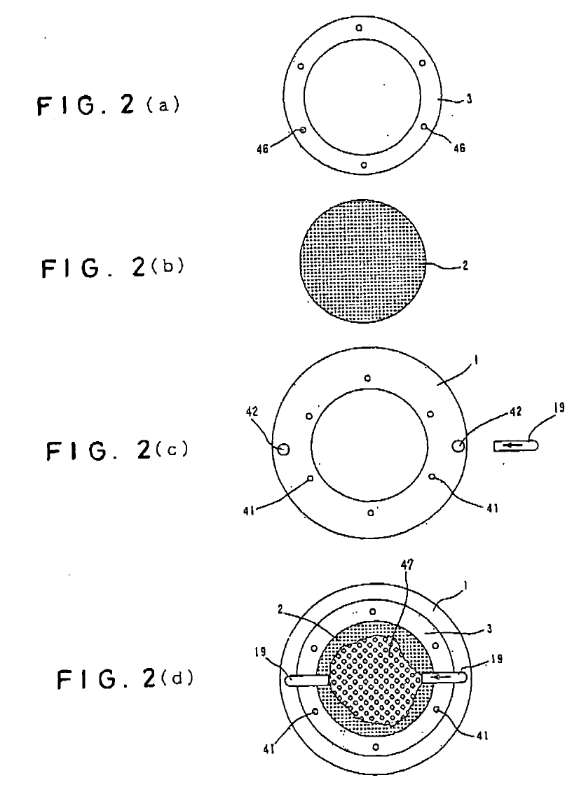

The bottom plate 1, test material 2, holder ring 3 and the like shown in FIG. 1 are constituted and assembled as shown in FIG. 2 and FIG. 3. To wit, the ring-shaped holder ring 3 provided with bolt holes 46 as shown in FIG. 2(a) is provided on its bottom surface with an O-ring 5 as the elastic ring as shown in FIG. 3. This O-ring 5 40 allows the test material 2 formed as shown in FIG. 2(b) to be sandwiched against the upper surface of the bottom plate 1. As a result, not only plate-shaped material but also material in the membrane, sponge, filter or other form can be easily secured to the bottom plate 1. In the example shown in FIG. 2(c), the bottom plate 1 is provided with screw holes 41 which correspond to the bolt holes 46 of the holder ring 3 and also the two access port openings 42 shown in the figure. In addition, the access ports 19 pass through these access port openings 42 so that blood 47 or the like can be easily introduced upon the test material 2 as shown in FIG. 2(d) which illustrates the state of these pieces of apparatus after assembly.

As shown in FIG. 3, the angle between the sides of the cone 16 at its vertex 43 is large and accordingly the angle between the cone 16 and the test material upon the bottom plate is small and thus formed in a state near that of a plate, and thus when the cone 16 rotates with the vertex 43 in proximity to the test material 2, the space above the test material 2 and below the bottom surface of the cone 16 can be made small so testing can be performed with a small amount of blood or the like. The cone 16 is placed upon the bottom plate receptacle 44 of the cone holder 13, and the ring 17 with screws around its periphery is tightened around the gasket 45 so that the blood or the like on the bottom surface of the cone 16 does not leak above.

The present invention is thus constituted as described above, so the blood or the like supplied upon the test material can be agitated by the computer at a stipulated speed by means of the cone of the cone-plate type shear stress loading apparatus. In addition, at the same time, the blood upon the test surface at this time can be observed by the upright incident light fluorescent microscope equipped with its objective lens provided above the transparent cone. The materials tested at this time may be not only plate-shaped materials but also materials having the shape of a membrane, sponge, filter or any other shape, and also the state of adhesion of blood cell components to opaque materials may be observed in the presence of shear stresses and in real time and quantitatively analyzed by an image analysis apparatus.

Moreover, the mechanism is simple consisting of only a cone that rotates above the test material, so the amount of blood required for a single material evaluation is only roughly several hundred microliters but evaluation can be performed accurately with this small amount of blood.

### INDUSTRIAL APPLICABILITY

The invention according to claim 1 of this application is a cone-plate type apparatus for evaluating compatibility with blood or the like characterized in comprising: a bottom plate that secures test material, a cone made of transparent material that rotates while in contact with blood or the like supplied upon the test material, a cone holder that secures the cone therein and that has a rotary driver on its periphery, a driving apparatus that rotates the cone holder via the rotary driver and an incident light microscope for observing blood or the like on the test material from above the cone, so it can provide a general-purpose observation apparatus that can be used to observe and quantitatively evaluate the interaction between blood cell components and not only plate-shaped materials but also materials having the shape of a membrane, sponge, filter or any other shape, along with opaque materials, in the presence of shear stresses and in real time.

In addition, the invention according to claim 2 is the cone-plate type apparatus for evaluating compatibility with blood or the like according to claim 1 characterized in that the rotary driver of the cone holder is a ring gear and the ring gear is rotary driven by a gear of the driving apparatus via an intermediate gear, so a cone driving mechanism can be easily formed without providing a driving apparatus above the cone.

In addition, the invention according to claim 3 is the cone-plate type apparatus for evaluating compatibility with blood or the like according to claim 2 characterized in that each of the gears is supported at a stipulated position via bearings, so each gear can be reliably supported at the stipulated position and also there is little driving resistance and the shear force at the cone can be measured accurately.

In addition, the invention according to claim 4 is the cone-plate type apparatus for evaluating compatibility with blood or the like according to claim 3 characterized in that the bearings are air bearings and an air compressor that supplies air to the air bearings is provided, so each gear can be supported in a state with virtually no friction and thus the measurement of the shear force at the cone can be measured more accurately.

The invention according to claim 5 is the cone-plate type apparatus for evaluating compatibility with blood or the like according to claim 1 characterized in that the test material upon the bottom plate is in the form of a plate, membrane, sponge, filter or any other shape and is secured by means of a ring member, so various types of test materials can be reliably secured.

The invention according to claim 6 is the cone-plate type apparatus for evaluating compatibility with blood or the like according to claim 5 characterized in that the ring member is provided with an elastic ring, so the test material can be reliably secured by the elastic ring.

The invention according to claim 7 is the cone-plate type apparatus for evaluating compatibility with blood or the like according to claim 6 characterized in that the elastic ring is an O-ring, so the test material can be reliably secured by a simple means.

The invention according to claim 8 is the cone-plate type apparatus for evaluating compatibility with blood or the like according to claim 6 characterized in that screws that screw into the bottom plate support member are provided, so the bottom plate can be easily attached to the bottom plate support member and also, by adjusting how far they are screwed in, the distance between the test material and the tip of the cone can be adjusted arbitrarily.

The invention according to claim 9 is the cone-plate type apparatus for evaluating compatibility with blood or the like according to claim 1 characterized in that access ports that are able to supply blood or the like to the surface of the test material in the presence of shear stress due to the rotation of the cone is provided, so it is possible to supply blood or the like in the state with the cone rotating and apply a load.

The invention according to claim 10 is the cone-plate type apparatus for evaluating compatibility with blood or the like according to claim 9 characterized in that the bottom plate has two holes for installing the access ports, so a means of supplying blood or the like in the state with the cone rotating can be easily attached.

The invention according to claim 11 is the cone-plate type apparatus for evaluating compatibility with blood or the like according to claim 1 characterized in comprising a cone that makes a small angle with respect to the test material so that the interaction between the test material and blood cell components can be examined with a small quantity of blood or the like, and thus the interaction between the test material and the blood cell components can be measured and observed with a small quantity of blood or the like.

The invention according to claim 12 is the cone-plate type apparatus for evaluating compatibility with blood or the like according to claim 1 characterized in comprising a height adjustment apparatus that adjusts the height of the cone with respect to the bottom plate, so it is possible to adjust the position of the cone tip with respect to the test material and thus accurate measurement and observation can be conducted.

The invention according to claim 13 is the cone-plate type apparatus for evaluating compatibility with blood or the like according to claim 1 characterized in that the objective lens of the microscope is disposed above the cone, so the state of the test material can be easily observed from above the cone.

The invention according to claim 14 is the cone-plate type apparatus for evaluating compatibility with blood or the like according to claim 1 characterized in comprising a securing ring that secures the cone to the cone holder, so the cone can be easily and reliably secured to the cone holder.

The invention according to claim 15 is the cone-plate type apparatus for evaluating compatibility with blood or the like according to claim 1 characterized in comprising a gasket provided between the cone and the cone holder, thus preventing leakage of blood or blood platelet suspension, so leakage of blood or the like can be reliably and easily prevented.

The invention according to claim 16 is the cone-plate type apparatus for evaluating compatibility with blood or the like according to claim 1 characterized in comprising a linkage screw provided between the cone holder and the motor that drives it, so the cone holder can be reliably driven.

The invention according to claim 17 is the cone-plate type apparatus for evaluating compatibility with blood or the like according to claim 1 characterized in comprising a moveable arm that secures the bottom plate and also rotatably supports the cone bolder such that the cone is able to oscillate freely between directly below the microscope and other portions, so the cone can be removed from the area directly below the microscope and various operations can be performed, and returned to the area directly below the microscope so that observation with the microscope can be performed easily.

The invention according to claim 18 is the cone-plate type apparatus for evaluating compatibility with blood or the like according to claim 1 characterized in comprising a translation apparatus that focuses the microscope on the surface of the test material provided in the driving apparatus area, so the microscope can be easily focused without performing operations on the microscope.

The invention according to claim 19 is the cone-plate type apparatus for evaluating compatibility with blood or the like according to claim 1 characterized in that the driving apparatus is provided with a motor that can load a superposition strain, so the motor that performs rotary driving of the cone can be accurately controlled.

The invention according to claim 20 is the cone-plate type apparatus for evaluating compatibility with blood or the like according to claim 1 characterized in comprising an X-Y-Z axial movement mechanism that changes the observation position on the surface of the test material, so the position of observation with the microscope can be easily moved to an arbitrary position.

The invention according to claim 21 is the cone-plate type apparatus for evaluating compatibility with blood or the like according to claim 1 characterized in that the microscope is an incident light fluorescent microscope provided with a super-long focal length objective lens for observing the surface of the test material from above, so that the surface of the test material can be easily observed by microscope from above.

The invention according to claim 22 is the cone-plate type apparatus for evaluating compatibility with blood or the like according to claim 1 characterized in that the microscope is provided with an illumination apparatus that projects light from the front side of the test material and an illumination apparatus that projects light from the rear side thereof, so it can be used as either an upright incident light fluorescent microscope or an inverted transmission microscope depending on the characteristics of the test materials.

The invention according to claim 23 is the cone-plate type apparatus for evaluating compatibility with blood or the like according to claim 1 characterized in comprising a box that entirely encloses the apparatus and that is able to control the interior to a fixed temperature, so accurate measurement and observation can be performed within the box.

The invention according to claim 24 is the cone-plate type apparatus for evaluating compatibility with blood or the like according to claim 1 characterized in comprising a temperature control apparatus for controlling the interior of a box to a fixed temperature, so the temperature can be controlled accurately by the temperature control apparatus and more accurate measurement and observation can be performed.

The invention according to claim 25 is the cone-plate type apparatus for evaluating compatibility with blood or the like according to claim 1 characterized in comprising a unitary vibration proof plate upon which the driving apparatus and microscope are installed, so the effect of vibration on the drive mechanism and microscope can be reduced and also the vibration modes of both can be made constant, and thus accurate measurement and observation of the number of particles of blood adhering to the material can be performed.

The invention according to claim 26 is the cone-plate type apparatus for evaluating compatibility with blood or the like according to claim 1 characterized in comprising an adjustment mechanism that adjusts the relative position of the observation apparatus consisting of the microscope and a shear stress measuring apparatus, so adjustment of the positions of the two pieces of apparatus is possible and accurate measurement and observation can be performed.

The invention according to claim 27 is the cone-plate type apparatus for evaluating compatibility with blood or the like according to claim 1 characterized in comprising a computer that controls the rotation of the cone, so the rotation of the cone can be reliably controlled to stipulated values.

The invention according to claim 28 is the cone-plate type apparatus for evaluating compatibility with blood or the like according to claim 1 characterized in that the incident light microscope comprises a super high-sensitivity camera, so the state of the blood or the like agitated by the cone can be accurately photographed.

The invention according to claim 29 is the cone-plate type apparatus for evaluating compatibility with blood or the like according to claim 1 characterized in that an image analyzer is connected to the incident light microscope, so images obtained by the microscope can be easily analyzed and that image data can be manipulated appropriately.

The invention according to claim 30 is the cone-plate type apparatus for evaluating compatibility with blood or the like according to claim 1 characterized in that a timer is connected to the incident light microscope, so images obtained by the microscope can be recorded at appropriate intervals, and the long-term state can be recorded.

The invention according to claim 31 is the cone-plate type apparatus for evaluating compatibility with blood or the like according to claim 1 characterized in that a video recorder is connected to the incident light microscope, so observation images obtained by the microscope can be recorded over long periods of time, and can be output when necessary and observed.

The invention according to claim 32 is the cone-plate type apparatus for evaluating compatibility with blood or the like according to claim 1 characterized in that a monitor is connected to the incident light microscope, so images obtained by the microscope can be viewed on the monitor, and can be easily observed.

1. A cone-plate type apparatus for evaluating compatibility with blood or the like characterized in comprising:
a bottom plate (1) that secures test material (2),
a cone (16) made of transparent material that rotates while in contact with blood or the like supplied upon said test material,
a cone holder (13) that secures said cone therein and that has a rotary driver (14) on its periphery,
a driving apparatus (8) that rotates the cone holder via said rotary driver and an incident light fluorescent microscope (20) for observing blood or the like on said test material from above said cone.

2. The cone-plate type apparatus for evaluating compatibility with blood or the like according to claim 1 characterized in that the rotary driver (14) of said cone holder (13) is a ring gear and said ring gear is rotary driven by a gear of said driving apparatus via an intermediate gear (12).

3. The cone-plate type apparatus for evaluating compatibility with blood or the like according to claim 2 characterized in that each of said gears (12) and (14) is supported at a stipulated position via bearings.

4. The cone-plate type apparatus for evaluating compatibility with blood or the like according to claim 3 characterized in that said bearings are air bearings and an air compressor (15) that supplies air to the air bearings is provided.

5. The cone-plate type apparatus for evaluating compatibility with blood or the like according to claim 1 characterized in that the test material upon said bottom plate is in a form of a plate, membrane, sponge or filter and is secured by means of ring members (3) and (5).

6. The cone-plate type apparatus for evaluating compatibility with blood or the like according to claim 5 characterized in that the ring member is provided with an elastic ring.

7. The cone-plate type apparatus for evaluating compatibility with blood or the like according to claim 6 characterized in that said elastic ring is an O-ring (5).

8. The cone-plate type apparatus for evaluating compatibility with blood or the like according to claim 1 characterized in that said bottom plate has a plurality of screw holes (41) in its periphery, and screws that screw in from a bottom surface are provided.

9. The cone-plate type apparatus for evaluating compatibility with blood or the like according to claim 1 characterized in that a plurality of access ports (19) that are able to supply blood or the like to a surface of the test material in the presence of shear stress due to the rotation of said cone is provided.

10. The cone-plate type apparatus for evaluating compatibility with blood or the like according to claim 9 characterized in that said bottom plate has a plurality of holes (42) for installing said access ports.

11. The cone-plate type apparatus for evaluating compatibility with blood or the like according to claim 1 characterized in that said cone (16) is a cone that makes a small angle with respect to the test material so that the interaction between the test material and blood cell components can be examined with a small quantity of blood or the like.

12. The cone-plate type apparatus for evaluating compatibility with blood or the like according to claim 1 characterized in comprising a height adjustment apparatus that adjusts a height of the cone with respect to said bottom plate.

13. The cone-plate type apparatus for evaluating compatibility with blood or the like according to claim 1 characterized in that said cone is disposed such that an objective lens (26) of the microscope is disposed above it.

14. The cone-plate type apparatus for evaluating compatibility with blood or the like according to claim 1 characterized in that said cone has a securing ring (17) that secures it to the cone holder.

15. The cone-plate type apparatus for evaluating compatibility with blood or the like according to claim 1 characterized in comprising a gasket (45) provided between said cone and said cone holder, thus preventing leakage of blood or blood platelet suspension.

16. The cone-plate type apparatus for evaluating compatibility with blood or the like according to claim 1 characterized in comprising a linkage screw provided between said cone holder (13) and said driving apparatus (8) that drives the cone holder.

17. The cone-plate type apparatus for evaluating compatibility with blood or the like according to claim 1 characterized in that a rotating shaft of said driving apparatus secures said bottom plate and also rotatably supports said cone holder such that the cone is able to move freely between directly below the microscope and other portions.

18. The cone-plate type apparatus for evaluating compatibility with blood or the like according to claim 1 characterized in comprising a height adjustment apparatus (22) that focuses the microscope on a surface of the test material and that is provided in an area of said driving apparatus.

19. The cone-plate type apparatus for evaluating compatibility with blood or the like according to claim 1 characterized in that said driving apparatus (8) is provided with a motor that can load a superposition strain.

20. The cone-plate type apparatus for evaluating compatibility with blood or the like according to claim 1 characterized in comprising X-Y-Z axial movement apparatus (22) and (23) that change an observation position on a surface of said test material.

21. The cone-plate type apparatus for evaluating compatibility with blood or the like according to claim 1 characterized in that said microscope (20) is an incident light fluorescent microscope provided with a super-long focal length objective lens (26) for observing a surface of said test material from above.

22. The cone-plate type apparatus for evaluating compatibility with blood or the like according to claim 1 characterized in that said microscope is provided with an illumination apparatus (27) that projects light from a front side of said test material and an illumination apparatus (35) that projects light from a rear side.

23. The cone-plate type apparatus for evaluating compatibility with blood or the like according to claim 1 characterized in comprising a box (38) that entirely encloses said apparatus and that is able to control its interior to a fixed temperature.

24. The cone-plate type apparatus for evaluating compatibility with blood or the like according to claim 1 characterized in comprising a temperature control apparatus (24) for controlling an interior of a box to a fixed temperature.

25. The cone-plate type apparatus for evaluating compatibility with blood or the like according to claim 1 characterized in comprising a unitary vibration proof plate upon which said driving apparatus (8) and microscope (20) are installed.

26. The cone-plate type apparatus for evaluating compatibility with blood or the like according to claim 1 characterized in comprising a height adjustment mechanism (22) that adjusts a relative position of an observation apparatus consisting of said microscope and a shear stress loading apparatus (40).

27. The cone-plate type apparatus for evaluating compatibility with blood or the like according to claim 1 characterized in comprising a computer (25) that controls the rotation of said cone.

28. The cone-plate type apparatus for evaluating compatibility with blood or the like according to claim 1 characterized in that said incident light microscope has a super high-sensitivity camera (30).

29. The cone-plate type apparatus for evaluating compatibility with blood or the like according to claim 1 characterized in that an image analyzer (31) is connected to said incident light microscope.

30. The cone-plate type apparatus for evaluating compatibility with blood or the like according to claim 1 characterized in that a timer (32) is connected to said incident light microscope.

31. The cone-plate type apparatus for evaluating compatibility with blood or the like according to claim 1 characterized in that a video recorder (33) is connected to said incident light microscope.

32. The cone-plate type apparatus for evaluating compatibility with blood or the like according to claim 1 characterized in that a monitor (34) is connected to said incident light microscope.
